(19) United States
(12) Patent Application Publication
(10) Pub. No.: US 2004/0106671 A1
(43) Pub. Date: Jun. 3, 2004

(54) Remedies for vesical hyperesthesia

(76) Inventors: Tsuyoshi Yamagata, Sunto-gun (JP); Kaoru Atsuki, Sunto-gun (JP); Tetsuji Ohno, Sunto-gun (JP); Shiro Shirakura, Mishima-shi (JP); Akira Karazawa, Sunto-gun (JP)

Correspondence Address: FITZPATRICK CELLA HARPER & SCINTO, 30 ROCKEFELLER PLAZA, NEW YORK, NY, 10112, US

(21) Appl. No.: 10/472,144

(22) PCT Filed: Mar. 29, 2002
(86) PCT No.: PCT/JP02/03168

(30) Foreign Application Priority Data
Mar. 30, 2001 (JP) 2001-099800

(51) Int. Cl. A61K 31/38; A61K 31/335; A61K 31/16

(52) U.S. Cl. 514/431; 514/450; 514/613

(57) ABSTRACT

The present invention provides a therapeutic agent for bladder hypersensitivity comprising, as an active ingredient, a tricyclic compound represented by formula (I):

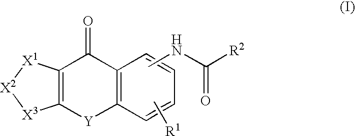

[wherein R¹ represents a hydrogen atom, substituted or unsubstituted lower alkyl, substituted or unsubstituted lower alkoxy or halogen;

X¹—X²—X³ represents CR⁵═CR⁶—CR⁷═CR⁸, N(O)ₘ═CR⁶—CR⁷═CR⁸, CR⁵═CR⁶—N(O)ₘ═CR⁸, CR⁵═CR⁶—CR⁷═N(O)ₘ, CR⁵═CR⁶—O, CR⁵═CR⁶—S, O—CR⁷═CR⁸, S—CR⁷═CR⁸ or O—CR⁷═N;

Y represents —CH₂S—, —CH₂SO—, —CH₂SO₂—, —CH₂O—, —CH═CH—, —(CH₂)ₚ—, —SCH₂—, —SOCH₂—, —SO₂CH₂— or —OCH₂—; and

R² represents a hydrogen atom, substituted or unsubstituted lower alkyl, substituted or unsubstituted lower alkenyl, substituted or unsubstituted lower alkoxy, amino, mono(substituted or unsubstituted lower alkyl)-substituted amino, di(substituted or unsubstituted lower alkyl)-substituted amino, substituted or unsubstituted aryl, substituted or unsubstituted heteroaryl, substituted or unsubstituted aralkylamino, substituted or unsubstituted arylamino or a substituted or unsubstituted heteroalicyclic group] or a pharmaceutically acceptable salt thereof.

### TECHNICAL FIELD

The present invention relates to a therapeutic agent for bladder hypersensitivity.

### BACKGROUND ART

Micturition reflex is physiologically controlled by the complex reflex pathways including peripheral and central nervous systems [Urology, Vol. 50, Supplement No. 6A, pp. 36-52 (1997)]. Detrusor overactivity is diagnosed by the observation of involuntary (uninhibited) detrusor contraction in cystometry of a patient with urinary frequency, urinary urge incontinence or urinary urgency. The detrusor overactivity is considered to be a main cause of urinary urge incontinence (that is, an involuntary loss of urine associated with a sudden and strong desire to void). The detrusor overactivity is also considered to be a main cause of urinary urgency, which can lead to urinary frequency. The detrusor overactivity is observed when the bladder is hypersensitive.

Bladder hypersensitivity is observed when a patient suffers from cystitis, hormone imbalance, benign prostatic hyperplasia, etc. A sensation of the bladder filling is transmitted to the central nerve via two sensory afferents, the A fibers and the C fibers; and the increase of C-fiber activity is involved in bladder hypersensitivity. Bladder hypersensitivity induces bladder pain, urinary urgency, urinary urge incontinence and urinary frequency.

Tricyclic compounds having the activity to prolong the intervals of bladder contractions and pharmaceutically acceptable salts thereof are known as therapeutic agents for urinary incontinence (WO97/14672 and WO98/46587). However, it is not known that the compound groups have an inhibitory activity on bladder hypersensitivity.

### DISCLOSURE OF THE INVENTION

The present invention relates to the following (1) to (27).

(1) A therapeutic agent for bladder hypersensitivity comprising, as an active ingredient, a tricyclic compound represented by formula (I):

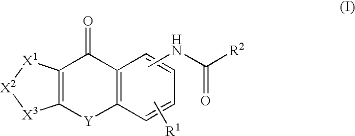

[wherein R¹ represents a hydrogen atom, substituted or unsubstituted lower alkyl, substituted or unsubstituted lower alkoxy or halogen;

X¹—X²—X³ represents CR⁵═CR⁶—CR⁷═CR⁸ (wherein R⁵, R⁶, R⁷ and R⁸, which may be the same or different, each represent a hydrogen atom, substituted or unsubstituted lower alkyl, hydroxy, substituted or unsubstituted lower alkoxy, nitro, amino, mono(lower alkyl)-substituted amino, di (lower alkyl)-substituted amino, substituted or unsubstituted lower alkanoylamino or halogen), N(O)ₘ═CR⁶—CR⁷═CR⁸ (wherein R⁶, R⁷ and R⁸ have the same significances as defined above, respectively, and m represents 0 or 0.1), CR⁵═CR⁶—N(O)ₘ═CR⁸ (wherein R⁵, R⁶, R⁸ and m have the same significances as defined above, respectively), CR⁵═CR⁶—CR⁷═N(O)ₘ (wherein R⁵, R⁶, R⁷ and m have the same significances as defined above, respectively), CR⁵═CR⁶—O (wherein R⁵ and R⁶ have the same significances as defined above, respectively), CR⁵═CR⁶—S (wherein R⁵ and R⁶ have the same significances as defined above, respectively), OCR⁷═CR⁸ (wherein R⁷ and R⁸ have the same significances as defined above, respectively), S—CR⁷═CR⁸ (wherein R⁷ and R⁸ have the same significances as defined above, respectively) or O—CR⁷═N (wherein R⁷ has the same significance as defined above);

Y represents —CH₂S—, —CH₂SO—, —CH₂SO₂—, —CH₂O—, —CH═CH—, —(CH₂)ₚ— (wherein p represents an integer of 0 to 2), —SCH₂—, —SOCH₂—, —SO₂CH₂— or —OCH₂—; and

R² represents a hydrogen atom, substituted or unsubstituted lower alkyl, substituted or unsubstituted lower alkenyl, substituted or unsubstituted lower alkoxy, amino, mono(substituted or unsubstituted lower alkyl)-substituted amino, di(substituted or unsubstituted lower alkyl)substituted amino, substituted or unsubstituted aryl, substituted or unsubstituted heteroaryl, substituted or unsubstituted aralkylamino, substituted or unsubstituted arylamino or a substituted or unsubstituted heteroalicyclic group] or a pharmaceutically acceptable salt thereof.

(2) A therapeutic agent for bladder hypersensitivity comprising, as an active ingredient, a tricyclic compound represented by formula (Ia):

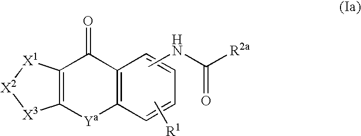

[wherein R¹ and X¹—X²—X³ have the same significances as defined above, respectively;

Y^{a} represents —CH₂SO₂—, —SCH₂—, —SOCH₂—, —SO₂CH₂— or OCH₂—; and

when Y^{a} is —CH₂SO₂—, —SCH₂—, —SOCH₂— or —SO₂CH₂—,

R^{2a} represents a hydrogen atom, substituted or unsubstituted lower alkyl, substituted or unsubstituted lower alkenyl, trifluoromethyl, substituted or unsubstituted lower alkoxy, amino, mono(substituted or unsubstituted lower alkyl)substituted amino, di(substituted or unsubstituted lower alkyl)-substituted amino, substituted or unsubstituted aryl, substituted or unsubstituted heteroaryl, substituted or unsubstituted aralkylamino, substituted or unsubstituted arylamino, a substituted or unsubstituted heteroalicyclic group or formula (II):

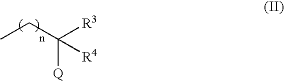

(wherein n is 0 or 1; R³ and R⁴, which may be the same or different, each represent a hydrogen atom, substituted or unsubstituted lower alkyl, substituted or unsubstituted cycloalkyl, substituted or unsubstituted aryl, substituted or unsubstituted aralkyl or trifluoromethyl, or R³ and R⁴ may be combined together with the adjacent carbon atom to form cycloalkyl; and Q represents hydroxy, substituted or unsubstituted lower alkoxy, amino or halogen), and when Y^{a} is —OCH₂—,

R^{2a} represents a hydrogen atom, substituted or unsubstituted lower alkenyl, trifluoromethyl, substituted or unsubstituted lower alkoxy, amino, mono(substituted or unsubstituted lower alkyl)substituted amino, di(substituted or unsubstituted lower alkyl)-substituted amino, substituted or unsubstituted aryl, substituted or unsubstituted heteroaryl, substituted or unsubstituted aralkylamino, substituted or unsubstituted arylamino, a substituted or unsubstituted heteroalicyclic group or formula (II):

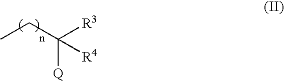

(wherein n, R³, R⁴ and Q have the same significances as defined above, respectively)] or a pharmaceutically acceptable salt thereof.

(3) The therapeutic agent for bladder hypersensitivity comprising, as an active ingredient, a tricyclic compound or a pharmaceutically acceptable salt thereof according to (2), wherein Y^{a} is —CH₂SO₂—, —SCH₂—, SOCH₂— or —SO₂CH₂—.

(4) The therapeutic agent for bladder hypersensitivity comprising, as an active ingredient, a tricyclic compound or a pharmaceutically acceptable salt thereof according to (2), wherein Y^{a} is —OCH₂—.

(5) The therapeutic agent for bladder hypersensitivity comprising, as an active ingredient, a tricyclic compound or a pharmaceutically acceptable salt thereof according to any of (2) to (4), wherein R¹ is a hydrogen atom, substituted or unsubstituted lower alkoxy or halogen.

(6) The therapeutic agent for bladder hypersensitivity comprising, as an active ingredient, a tricyclic compound or a pharmaceutically acceptable salt thereof according to any of (2) to (4), wherein R¹ is a hydrogen atom.

(7) The therapeutic agent for bladder hypersensitivity comprising, as an active ingredient, a tricyclic compound or a pharmaceutically acceptable salt thereof according to any of (2), (5) and (6), wherein Y^{a} is —CH₂SO₂—, —SO₂CH₂— or —OCH₂—.

(8) The therapeutic agent for bladder hypersensitivity comprising, as an active ingredient, a tricyclic compound or a pharmaceutically acceptable salt thereof according to any of (2), (5) and (6), wherein Y^{a} is 30 CH₂SO₂— or —SO₂CH₂—.

(9) The therapeutic agent for bladder hypersensitivity comprising, as an active ingredient, a tricyclic compound or a pharmaceutically acceptable salt thereof according to any of (2), (5) and (6), wherein Y^{a} is —CH₂SO₂—.

(10) The therapeutic agent for bladder hypersensitivity comprising, as an active ingredient, a tricyclic compound or a pharmaceutically acceptable salt thereof according to any of (2) to (9), wherein X¹—X²—X³ is S—CR⁷═CR⁸ (wherein R⁷ and R⁸ have the same significances as defined above, respectively).

(11) The therapeutic agent for bladder hypersensitivity comprising, as an active ingredient, a tricyclic compound or a pharmaceutically acceptable salt thereof according to any of (2) to (9), wherein X¹—X²—X³ is CR⁵═CR⁶—CR⁷═CR⁸ (wherein R⁵, R⁶, R⁷ and R⁸ have the same significances as defined above, respectively).

(12) The therapeutic agent for bladder hypersensitivity comprising, as an active ingredient, a tricyclic compound or a pharmaceutically acceptable salt thereof according to any of (2) to (11), wherein R^{2a} is formula (II):

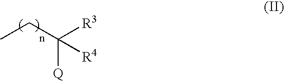

(wherein n, R³, R⁴ and Q have the same significances as defined above, respectively).

(13) The therapeutic agent for bladder hypersensitivity comprising, as an active ingredient, a tricyclic compound or a pharmaceutically acceptable salt thereof according to (12), wherein n is 0.

(14) The therapeutic agent for bladder hypersensitivity comprising, as an active ingredient, a tricyclic compound or a pharmaceutically acceptable salt thereof according to (13), wherein R³ is methyl, R⁴ is trifluoromethyl, and Q is hydroxy.

(15) The therapeutic agent for bladder hypersensitivity comprising, as an active ingredient, a tricyclic compound or a pharmaceutically acceptable salt thereof according to (2), wherein R¹ is a hydrogen atom, Y^{a} is —CH₂SO₂—, X¹—X²—X³ is S—CR⁷═CR⁸ (wherein R⁷ and R⁸ have the same significances as defined above, respectively), and R² is formula (III):

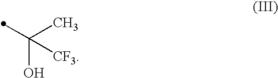

(16) A therapeutic agent for bladder hypersensitivity comprising, as an active ingredient, a tricyclic compound represented by formula (Ib):

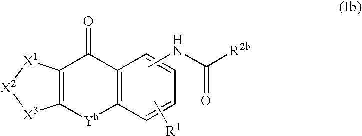

[wherein R¹ and X¹—X²—X³ have the same significances as defined above, respectively;

Y^{b} represents —CH₂O—, —CH₂S—, —CH₂SO—, —CH═CH— or —(CH₂)ₚ— (wherein p has the same significance as defined above); and R^{2b} represents formula (III):

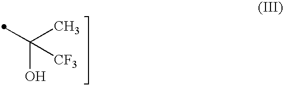

or a pharmaceutically acceptable salt thereof.

(17) The therapeutic agent for bladder hypersensitivity comprising, as an active ingredient, a tricyclic compound or a pharmaceutically acceptable salt thereof according to (16), wherein X¹—X²—X³ is CR⁵═CR⁶—CR⁷═CR⁸ (wherein R⁵, R⁶, R⁷ and R⁸ have the same significances as defined above, respectively) or CR⁵═CR⁶—CR⁷═N (wherein R⁵, R⁶ and R⁷ have the same significances as defined above, respectively).

(18) The therapeutic agent for bladder hypersensitivity comprising, as an active ingredient, a tricyclic compound or a pharmaceutically acceptable salt thereof according to (16), wherein X¹—X²—X³ is CR⁵═CR⁶—O (wherein R⁵ and R⁶ have the same significances as defined above, respectively) or CR⁵═CR⁶—S (wherein R⁵ and R⁶ have the same significances as defined above, respectively).

(19) The therapeutic agent for bladder hypersensitivity comprising, as an active ingredient, a tricyclic compound or a pharmaceutically acceptable salt thereof according to (16), wherein X¹—X²—X³ is O—CR⁷═CR⁸ (wherein R⁷ and R⁸ have the same significances as defined above, respectively) or S—CR⁷═CR⁸ (wherein R⁷ and R⁸ have the same significances as defined above, respectively).

(20) The therapeutic agent for bladder hypersensitivity comprising, as an active ingredient, a tricyclic compound or a pharmaceutically acceptable salt thereof according to any of (16) to (19), wherein Y^{b} is —CH₂O—.

(21) The therapeutic agent for bladder hypersensitivity comprising, as an active ingredient, a tricyclic compound or a pharmaceutically acceptable salt thereof according to any of (16) to (19), wherein Y^{b} is (CH₂)ₚ— (wherein p has the same significance as defined above)

(22) The therapeutic agent for bladder hypersensitivity comprising, as an active ingredient, a tricyclic compound or a pharmaceutically acceptable salt thereof according to (21), wherein p is 0.

(23) The therapeutic agent for bladder hypersensitivity comprising, as an active ingredient, a tricyclic compound or a pharmaceutically acceptable salt thereof according to (21), wherein p is 2.

(24) The therapeutic agent for bladder hypersensitivity comprising, as an active ingredient, a tricyclic compound or a pharmaceutically acceptable salt thereof according to any of (16) to (19), wherein Y^{b} is CH═CH—.

(25) The therapeutic agent for bladder hypersensitivity comprising, as an active ingredient, a tricyclic compound or a pharmaceutically acceptable salt thereof according to any of (16) to (19), wherein Y^{b} is —CH₂S— or —CH₂SO—.

(26) Use of the tricyclic compound or the pharmaceutically acceptable salt thereof according to any of (1) to (25) for the production of a therapeutic agent for bladder hypersensitivity.

(27) A method for treating bladder hypersensitivity, comprising a step of administering an effective amount of the tricyclic compound or the pharmaceutically acceptable salt thereof according to any of (1) to (25).

Hereinafter, the compounds represented by formula (I) are referred to as Compounds (I), and the same applies to the compounds of other formula numbers.

In the definitions of the groups in formula (I), the lower alkyl moiety of the lower alkyl, the lower alkoxy, the mono(lower alkyl)-substituted amino and the di(lower alkyl)-substituted amino includes straight-chain or branched lower alkyl groups having 1 to 8 carbon atoms, such as methyl, ethyl, propyl, isopropyl, butyl, isobutyl, sec-butyl, tert-butyl, pentyl, hexyl, 1,2,2-trimethylpropyl, heptyl and octyl. The two lower alkyl moieties of the di(lower alkyl)-substituted amino may be the same or different.

The lower alkanoyl moiety of the lower alkanoylamino includes lower alkanoyl groups having 1 to 6 carbon atoms, such as formyl, acetyl, propanoyl, butanoyl, pentanoyl, 2,2-dimethylpropanoyl and hexanoyl.

The lower alkenyl includes straight-chain or branched lower alkenyl groups having 2 to 6 carbon atoms, such as vinyl, allyl, 1-propenyl, methacryl, 1-butenyl, crotyl, pentenyl and hexenyl.

The aryl and the aryl moiety of the arylamino include aryl groups having 6 to 14 carbon atoms, such as phenyl, naphthyl and anthranyl.

The heteroaryl includes 5- or 6-membered monocyclic heteroaromatic groups containing at least one atom selected from the group consisting of a nitrogen atom, an oxygen atom and a sulfur atom, and bicyclic or tricyclic condensed heteroaromatic groups in which 3- to 8-membered rings are condensed and which contain at least one atom selected from the group consisting of a nitrogen atom, an oxygen atom and a sulfur atom. Specific examples are pyridyl, furyl, thienyl, quinolyl, imidazolyl, benzimidazolyl, thiazolyl, pyrazinyl, pyrimidinyl, pyridazinyl, isoquinolinyl, phthalazinyl, quinazolinyl, quinoxalinyl, naphthyridinyl, cinnolinyl, pyrrolyl, pyrazolyl, triazolyl, tetrazolyl, oxazolyl, indolyl, benzotriazolyl, benzothiazolyl, benzoxazolyl, purinyl, and the like.

The aralkyl moiety of the aralkylamino includes aralkyl groups having 7 to 12 carbon atoms, such as benzyl, phenethyl and naphthylmethyl.

The heteroalicyclic group includes 3- to 8-membered monocyclic heteroalicyclic groups containing at least one atom selected from the group consisting of a nitrogen atom, an oxygen atom and a sulfur atom, and bicyclic or tricyclic condensed heteroalicyclic groups in which 3- to 8-membered rings are condensed and which contain at least one atom selected from the group consisting of a nitrogen atom, an oxygen atom and a sulfur atom. Specific examples are tetrahydropyridinyl, tetrahydroquinolinyl, tetrahydroisoquinolinyl, tetrahydropyranyl, tetrahydrofuranyl, dihydrobenzofuranyl, pyrrolidinyl, piperidino, piperidyl, perhydroazepinyl, perhydroazocinyl, morpholino, morpholinyl, thiomorpholino, thiomorpholinyl, piperazinyl, homopiperidino, homopiperazinyl, dioxolanyl, imidazolidinyl, imidazolinyl, pyrazolidinyl, indolinyl, isoindolinyl, pyrrolinyl, pyrrolidonyl, piperidonyl, perhydroazepinonyl, thiazolidonyl, oxazolidonyl, succinimido, phthalimido, glutarimido, maleimido, hydantoinyl, thiazolidinedionyl, oxazolidinedionyl, tetrahydrothienyl, chromanyl, pipecolinyl, and the like.

The halogen means a fluorine, chlorine, bromine or iodine atom.

The substituted lower alkyl, the substituted lower alkoxy, the mono (substituted lower alkyl)-substituted amino, the di(substituted lower alkyl)-substituted amino, the substituted lower alkanoylamino and the substituted lower alkenyl each have 1 to a substitutable number (preferably 1 to 6, more preferably 0.1 to 4) of substituents which are the same or different. Examples of the substituents are hydroxy, halogen, nitro, amino, carboxy, mono(lower alkyl)-substituted amino, di(lower alkyl)-substituted amino, lower alkoxy, cycloalkyl, substituted cycloalkyl [the substituted cycloalkyl has 1 to 3 substituents which are the same or different, such as hydroxy, halogen, nitro, amino, mono(lower alkyl)substituted amino, di(lower alkyl)-substituted amino or lower alkoxy], aryl, substituted aryl (the substituent in the substituted aryl has the same significance as that in the substituted aryl described below), aralkyl, substituted aralkyl (the substituent in the substituted aralkyl has the same significance as that in the substituted aralkyl described below), substituted lower alkoxy [the substituted lower alkoxy has 1 to 3 substituents which are the same or different, such as hydroxy, halogen, nitro, amino, mono(lower alkyl)substituted amino, di(lower alkyl)-substituted amino or lower alkoxy], and the like. In the above, the cycloalkyl may be bound to the substituted lower alkyl by spiro-union. Herein, the halogen has the same significance as defined above, the lower alkyl moiety of the mono(lower alkyl)substituted amino, the di(lower alkyl)-substituted amino and the lower alkoxy has the same significance as the above-described lower alkyl, and the aryl has the same significance as defined above. The cycloalkyl includes cycloalkyl groups having 3 to 8 carbon atoms, such as cyclopropyl, cyclobutyl, cyclopentyl, cyclohexyl, cycloheptyl and cyclooctyl. The aralkyl includes aralkyl groups having 7 to 12 carbon atoms, such as benzyl, phenethyl and naphthylmethyl.

The substituted aryl, the substituted heteroaryl, the substituted aralkylamino and the substituted arylamino each have 1 to 3 substituents which are the same or different. Examples of the substituents are lower alkyl, hydroxy, amino, halogen, and the like, and the lower alkyl and the halogen have the same significances as defined above, respectively.

The substituted heteroalicyclic group has 1 to 3 substituents which are the same or different. Examples of the substituents are lower alkyl, hydroxy, halogen, and the like, and the lower alkyl and the halogen have the same significances as defined above, respectively.

In the definitions of formula (Ia) and formula (Ib), the lower alkyl moiety of the lower alkyl, the lower alkoxy, the mono(lower alkyl)-substituted amino and the di(lower alkyl)-substituted amino includes straight-chain or branched lower alkyl groups having 1 to 6 carbon atoms, such as methyl, ethyl, propyl, isopropyl, butyl, isobutyl, sec-butyl, tert-butyl, pentyl, hexyl and 1,2,2-trimethylpropyl. The two lower alkyl moieties of the di(lower alkyl)-substituted amino may be the same or different.

The halogen, the lower alkenyl, the aryl moiety of the aryl and the arylamino, the heteroaryl, the aralkyl moiety of the aralkyl and the aralkylamino, the heteroalicyclic group and the cycloalkyl respectively have the same significances as the halogen, the lower alkenyl, the aryl, the heteroaryl, the aralkyl, the heteroalicyclic group and the cycloalkyl in the definitions of the groups in formula (I) or in the definitions of the substituents in the definitions of the groups in formula (I).

The substituted lower alkyl, the substituted lower alkoxy, the mono(substituted lower alkyl)-substituted amino, the di(substituted lower alkyl)-substituted amino, the substituted lower alkenyl and the substituted cycloalkyl each have 1 to 3 substituents which are the same or different. Examples of the substituents are hydroxy, halogen, nitro, amino, carboxy, mono(lower alkyl)-substituted amino, di (lower alkyl)-substituted amino, lower alkoxy, and the like. The halogen has the same significance as defined above, and the lower alkyl moiety of the mono(lower alkyl)-substituted amino, the di(lower alkyl)-substituted amino and the lower alkoxy has the same significance as the above-described lower alkyl.

The substituted aryl, the substituted heteroaryl, the substituted aralkyl, the substituted aralkylamino and the substituted arylamino each have 1 to 3 substituents which are the same or different. Examples of the substituents are lower alkyl, hydroxy, amino, halogen, and the like, and the lower alkyl and the halogen have the same significances as defined above, respectively.

The substituted heteroalicyclic group has 1 to 3 substituents which are the same or different. Examples of the substituents are lower alkyl, hydroxyl, halogen, and the like, and the lower alkyl and the halogen have the same significances as defined above, respectively.

The pharmaceutically acceptable salts of Compounds (I), Compound (Ia) and Compound (Ib) include pharmaceutically acceptable acid addition salts, metal salts, ammonium salts, organic amine addition salts and amino acid addition salts. Examples of the acid addition salts are inorganic acid addition salts such as hydrochloride, hydrobromide, hydroiodide, nitrate, sulfate and phosphate, and organic acid addition salts such as formate, acetate, benzoate, maleate, fumarate, succinate, tartrate, citrate, oxalate, glyoxylate, methanesulfonate, ethanesulfonate, benzenesulfonate and lactate. Examples of the metal salts are alkali metal salts such as a lithium salt, a sodium salt and a potassium salt, alkaline earth metal salts such as a magnesium salt and a calcium salt, an aluminum salt, a zinc salt, and the like. Examples of the ammonium salts are ammonium, tetramethylammonium, and the like. Examples of the organic amine addition salts are salts with morpholine, piperidine, or the like, and examples of the amino acid addition salts are salts with glycine, phenylalanine, aspartic acid, glutamic acid, lysine, or the like.

The tricyclic compounds used in the present invention can be produced according to the methods disclosed in the above publications or similar methods, and can be isolated and purified by purification methods conventionally used in synthetic organic chemistry, for example, neutralization, filtration, extraction, washing, drying, concentration, recrystallization and various kinds of chromatography.

When it is desired to obtain a salt of the tricyclic compound used in the present invention, in the case where it is produced in the form of the salt, it can be subjected to purification as such, and where it is produced in the form of a free base, it can be converted into a salt, after being dissolved or suspended in a suitable solvent, by adding an acid or a base thereto.

There may be optical isomers for some of the tricyclic compounds used in the present invention. All possible stereoisomers and mixtures thereof can be used as active ingredients of the therapeutic agent of the present invention.

The tricyclic compounds or pharmaceutically acceptable salts thereof used in the present invention may exist in the form of adducts with water or various solvents, which can also be used as active ingredients of the therapeutic agent of the present invention.

The pharmacological activities of typical Compound (I) are described in test examples. In Test Examples 1-2, (S)-(+)-N-(5,5-dioxido-10-oxo-4,10-dihydrothieno[3,2-c][1]benzothiepin-9-yl)-3,3,3-trifluoro-2-hydroxy-2-methylpropanamide was used as a test compound. Hereinafter, the above compound is referred to as Compound 1. Compound 1 is the same compound as Compound (1-25) described in WO98/46587.

The pharmacological activities of Compound (I) are described in the following test examples.

### TEST EXAMPLE 1

### Inhibitory Activity on Hypersensitivity of Bladder with Cystitis

The experiment was carried out by referring to the method of Morikawa, et al. [Jpn. J. Pharmacol., Vol. 52, pp. 587-595 (1990)].

Male SD rats weighing from 250 to 400 g (supplied by Japan SLC) were used in the test. Five to seven animals of these rats were put in each metal cage and reared by allowing them to freely take commercially available chow and water, in an animal room at room temperature between 19 and 25° C. and humidity between 3.0 and 70% under illumination for 12 hours (from 7:00 a.m. to 7:00 p.m.) per day.

The rats were subjected to bladder catheterization. Under anesthesia with pentobarbital sodium, the bladder was exposed by midline incision of the abdomen. A polyethylene tube (PE-SO, Nippon Becton Dickinson Co., Ltd.), which had a blunt end to protect tissue from injury, was filled with a physiological saline (Otsuka Pharmaceutical Co., Ltd.) and inserted from the bladder apex. The bladder catheter was fixed with a surgical silk ligature and indwelled. The other end of the catheter was exposed subcutaneously from the back of the neck, plugged and then fixed to the skin with a surgical thread.

Two to three days after the bladder catheterization, a cystometry test was performed. The rats were put in a Bollman cage (Natsume Seisakusho Co., Ltd.) and a three-way cock was connected to the bladder catheter, one end of the cock was connected to a pressure transducer (Nihon Kohden Corp.) and the other end was connected to a 50-mL syringe (Terumo Corp.) arranged to an infusion pump (Harvard Apparatus, Inc.) for physiological saline infusion. The intravesical pressure signal from the pressure transducer was amplified with a strain pressure amplifier (AP-601G, Nihon Kohden Corp.) connected thereto, and was recorded on a thermal array recorder (RTA-1200, Nihon Kohden Corp.) via a polygraph system (RMP-6008, Nihon Kohden Corp.) containing the above amplifier. After the completion of the preparation for the measurement, a room temperature physiological saline was continuously infused into the bladder at a flow rate of 3 mL/h, and the intravesical pressure was measured to observe micturition intervals. After the micturition intervals became constant, the intravesical pressure waveform was recorded for one hour. Then, saline infusion was stopped, and 0.5 mL of xylene (Wako Pure Chemical Industries, Ltd.) was infused into the bladder through the catheter and was removed about 10 seconds later to cause cystitis. After 30 minutes, the continuous infusion of saline was started again and the intravesical pressure was monitored for about one hour to obtain the value before dosing. The test compound was suspended in a 0.5 w/v % aqueous solution of methylcellulose at a concentration of 0.05 mg/mL. The suspension or a vehicle was orally administered to the animals at a volume of 2 mL/kg. After the administration of the test compound, the intravesical pressure waveform was recorded for 4.5 hours. The period of 1, 2, 3 and 4 hours after the dosing was used as measuring time after the administration of the vehicle or the compound tested, and each measuring period included during a duration of 30 minutes before and after each measuring time i.e. from 30 to 90 minutes, from 90 to 150 minutes, from 150 to 210 minutes and from 210 to 270 minutes after the administration.

Pre-micturition contraction was measured as an index of bladder hypersensitivity. The values of pre-micturition contractions were read from the intravesical pressure waveform recorded on a chart paper using a digitizer (KD3220, Graphtec Corporation) controlled by a computer (PC-9801NS/R, NEC), and saved as a WJ2 file on Lotus 1-2-3 R2.5J (Lotus). The WJ2 file was taken into Excel for Windows version 7.0 (Microsoft). The amplitude of pre-micturition contractions was expressed as a relative value when the value before the drug administration that was defined as 100, and the average ±standard error was calculated for each group.

The values (%) of pre-micturition contractions after the administration of the vehicle or test compound are shown in Table 1.

TABLE 1 (1)

| | Control | Compound 1 |
|---|---|---|
| Before administration | 100.0 ± 0.0 | 100.0 ± 0.0 |
| After 1 hour | 86.6 ± 4.3 | 71.5 ± 6.5 |
| After 2 hours | 81.3 ± 4.3 | 64.7 ± 6.8* |
| After 3 hours | 80.5 ± 5.6 | 61.7 ± 6.8* |
| After 4 hours | 83.9 ± 5.9 | 61.6 ± 5.4* |

*p < 0.05 (comparison with the control group) [n = 24 (control group) and 13 (compound-administered group); Student's t-test]

In Test Example 1, Compound 1 inhibited pre-micturition contractions, which were irregular without voiding. Therefore, the results suggest that Compound 1 is useful as a therapeutic agent for bladder hypersensitivity by inhibiting the pre-micturition contractions (inhibiting the detrusor overactivity).

From the above result, Compounds (I) and pharmaceutically acceptable salts thereof are considered to be useful as therapeutic agents for bladder hypersensitivity.

### TEST EXAMPLE 2

### Acute Toxicity Test

The test compound was administered orally or intraperitoneally to 3 animals per group of dd male mice (body weight: 20±1 g). The minimum lethal dose (MLD) value was obtained by observing mortality on the seventh day after the administration.

As a result, MLD of Compound 1 was >1000 mg/kg by orally administration.

Compounds (I) and pharmaceutically acceptable salts thereof can be used as such or in various pharmaceutical forms. The pharmaceutical compositions of the present invention can be produced by uniformly mixing an effective amount of Compound (I) or a pharmaceutically acceptable salt thereof, as an active ingredient, with a pharmaceutically acceptable carrier. It is preferable that these pharmaceutical compositions are in a unit dose form suitable for administration such as oral administration or parenteral administration (including intravenous administration).

In the preparation of compositions for oral administration, any useful pharmaceutically acceptable carriers can be used. For example, liquid preparations for oral administration such as suspensions and syrups can be produced using water, sugars such as sucrose, sorbitol and fructose, glycols such as polyethylene glycol and propylene glycol, oils such as sesame oil, olive oil and soybean oil, antiseptics such as p-hydroxybenzoates, flavors such as strawberry flavor and peppermint, or the like. Capsules, tablets, powders and granules can be produced using excipients such as lactose, glucose, sucrose and mannitol, disintegrators such as starch and sodium alginate, lubricants such as magnesium stearate and talc, binders such as polyvinyl alcohol, hydroxypropyl cellulose and gelatin, surfactants such as fatty acid esters, plasticizers such as glycerin, or the like. Tablets and capsules are the most useful unit dose forms for oral administration because of the easiness of administration. Solid pharmaceutical carriers are used for the production of tablets and capsules.

Injections can be prepared using, for example, carriers comprising distilled water, a salt solution, a glucose solution or a mixture of salt water and a glucose solution. They are prepared as solutions, suspensions or dispersed solutions using appropriate auxiliaries according to conventional methods.

Compounds (I) or pharmaceutically acceptable salts thereof can be administered orally in the above pharmaceutical forms or parenterally as an injection or the like. The effective dose and administration schedule vary depending upon the mode of administration, the age, body weight and condition of a patient, or the like, but they are usually administered in a dose of 1 to 900 mg/60 kg/day, preferably 1 to 200 mg/60 kg/day.

Certain embodiments of the present invention are illustrated in the following examples.

### BEST MODES FOR CARRYING OUT THE INVENTION

### EXAMPLE 1

### Tablets

Tablets having the following compositions were prepared according to a conventional method.

Compound 1 (250 g), mannitol (1598.5 g), sodium starch glycolate (100 g), light silicic acid anhydride (10 g), magnesium stearate (40 g) and yellow iron oxide (1.5 g) were mixed according to a conventional method. The resulting mixture was compressed using a tableting machine within 8 mm diameter punch and die (Purepress Correct-12, Kikusui Seisakusho Ltd.) to prepare tablets each containing 25 mg of the active ingredient.

The formulation is shown in Table 2.

TABLE 2

| | | |
|---|---|---|
| Formulation | Compound 1 | 25 mg |
| | Mannitol | 159.85 mg |
| | Sodium starch glycolate | 10 mg |
| | Light silicic acid anhydride | 1 mg |
| | Magnesium stearate | 4 mg |
| | Yellow iron oxide | 0.15 mg |
| | | 200 mg |

### EXAMPLE 2

### Capsules

Capsules having the following composition were prepared according to a conventional method.

Compound 1 (500 g), lactose (300 g), light silicic acid anhydride (100 g) and sodium lauryl sulfate (100 g) were mixed according to a conventional method. The resulting mixture was encapsulated in hard capsules No. 1 (content: 100 mg/capsule) using a capsule filler (LZ-64, Zanasi) to prepare capsules each containing 50 mg of the active ingredient.

The formulation is shown in Table 3.

TABLE 3

| | | |
|---|---|---|
| Formulation | Compound 1 | 50 mg |
| | Lactose | 30 mg |
| | Light silicic acid anhydride | 10 mg |
| | Sodium lauryl sulfate | 10 mg |
| | | 100 mg |

### EXAMPLE 3

### Injection

An injection having the following composition is prepared according to a conventional method.

Compound 1 (1 g) is dissolved in 100 g of purified soybean oil, and 12 g of purified egg yolk lecithin and 25 g of glycerin for injection are added thereto. The resulting mixture is made up to 1000 mL with distilled water for injection, kneaded and emulsified according to a conventional method. The obtained dispersed solution is aseptically filtered using a 0.2 μm disposable membrane filter and aseptically packed in glass vials in 2 mL portions to prepare an injection containing 2 mg of the active ingredient per vial.

The formulation is shown in Table 4.

TABLE 4

| | | |
|---|---|---|
| Formulation | Compound 1 | 2 mg |
| | Purified soybean oil | 200 mg |
| | Purified egg yolk lecithin | 24 mg |
| | Glycerin for injection | 50 mg |
| | Distilled water for injection | 1.72 mL |
| | | 2.00 mL |

### INDUSTRIAL APPLICABILITY

The present invention provides a therapeutic agent for bladder hypersensitivity comprising, as an active ingredient, a tricyclic compound or a pharmaceutically acceptable salt thereof.

1. A therapeutic agent for bladder hypersensitivity comprising, as an active ingredient, a tricyclic compound represented by formula (I): 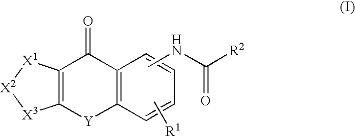
[wherein R¹ represents a hydrogen atom, substituted or unsubstituted lower alkyl, substituted or unsubstituted lower alkoxy or halogen;
X¹—X²—X³ represents CR⁵═CR⁶—CR⁷═CR⁸ (wherein R⁵, R⁶, R⁷ and R⁸, which may be the same or different, each represent a hydrogen atom, substituted or unsubstituted lower alkyl, hydroxy, substituted or unsubstituted lower alkoxy, nitro, amino, mono(lower alkyl)-substituted amino, di(lower alkyl)-substituted amino, substituted or unsubstituted lower alkanoylamino or halogen), N(O)ₘ═CR⁶—CR⁷═CR⁸ (wherein R⁶, R⁷ and R⁸ have the same significances as defined above, respectively, and m represents 0 or 1), CR⁵═CR⁶—N(O)ₘ═CR⁸ (wherein R⁵, R⁶, R⁸ and m have the same significances as defined above, respectively), CR⁵═CR⁶—CR⁷═N(O)ₘ (wherein R⁵, R⁶, R⁷ and m have the same significances as defined above, respectively), CR⁵═CR⁶—O (wherein R⁵ and R⁶ have the same significances as defined above, respectively), CR⁵═CR⁶—S (wherein R⁵ and R⁶ have the same significances as defined above, respectively), O—CR⁷═CR⁸ (wherein R⁷ and R⁸ have the same significances as defined above, respectively), SCR⁷═CR⁸ (wherein R⁷ and R⁸ have the same significances as defined above, respectively) or O—CR⁷═N (wherein R⁷ has the same significance as defined above);
Y represents —CH₂S—, —CH₂SO—, —CH₂SO₂—, —CH₂O—, —CH═CH—, —(CH₂)ₚ— (wherein p represents an integer of 0 to 2), —SCH₂—, —SOCH₂—, —SO₂CH₂— or —OCH₂—; and
R² represents a hydrogen atom, substituted or unsubstituted lower alkyl, substituted or unsubstituted lower alkenyl, substituted or unsubstituted lower alkoxy, amino, mono(substituted or unsubstituted lower alkyl)substituted amino, di(substituted or unsubstituted lower alkyl)-substituted amino, substituted or unsubstituted aryl, substituted or unsubstituted heteroaryl, substituted or unsubstituted aralkylamino, substituted or unsubstituted arylamino or a substituted or unsubstituted heteroalicyclic group] or a pharmaceutically acceptable salt thereof.

2. A therapeutic agent for bladder hypersensitivity comprising, as an active ingredient, a tricyclic compound represented by formula (Ia): 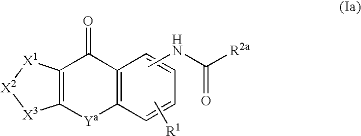
[wherein R¹ and X¹—X²—X³ have the same significances as defined above, respectively;
Y^{a} represents —CH₂SO₂—, —SCH₂—, —SOCH₂—, —SO₂CH₂— or —OCH₂—; and
when Y^{a} is —CH₂SO₂—, —SCH₂—, —SOCH₂— or —SO₂CH₂—,
R^{2a} represents a hydrogen atom, substituted or unsubstituted lower alkyl, substituted or unsubstituted lower alkenyl, trifluoromethyl, substituted or unsubstituted lower alkoxy, amino, mono(substituted or unsubstituted lower alkyl)-substituted amino, di(substituted or unsubstituted lower alkyl)-substituted amino, substituted or unsubstituted aryl, substituted or unsubstituted heteroaryl, substituted or unsubstituted aralkylamino, substituted or unsubstituted arylamino, a substituted or unsubstituted heteroalicyclic group or formula (II): 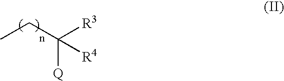
(wherein n is 0 or 1; R³ and R⁴, which may be the same or different, each represent a hydrogen atom, substituted or unsubstituted lower alkyl, substituted or unsubstituted cycloalkyl, substituted or unsubstituted aryl, substituted or unsubstituted aralkyl or trifluoromethyl, or R³ and R⁴ may be combined together with the adjacent carbon atom to form cycloalkyl; and Q represents hydroxy, substituted or unsubstituted lower alkoxy, amino or halogen), and
when Y^{a} is —OCH₂—,
R^{2a} represents a hydrogen atom, substituted or unsubstituted lower alkenyl, trifluoromethyl, substituted or unsubstituted lower alkoxy, amino, mono(substituted or unsubstituted lower alkyl)-substituted amino, di(substituted or unsubstituted lower alkyl)-substituted amino, substituted or unsubstituted aryl, substituted or unsubstituted heteroaryl, substituted or unsubstituted aralkylamino, substituted or unsubstituted arylamino, a substituted or unsubstituted heteroalicyclic group or formula (II): 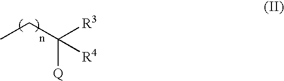
(wherein n, R³, R⁴ and Q have the same significances as defined above, respectively)] or a pharmaceutically acceptable salt thereof.

3. The therapeutic agent for bladder hypersensitivity comprising, as an active ingredient, a tricyclic compound or a pharmaceutically acceptable salt thereof according to claim 2, wherein Y^{a} is —CH₂SO₂—, —SCH₂—, —SOCH₂— or —SO₂CH₂—.

4. The therapeutic agent for bladder hypersensitivity comprising, as an active ingredient, a tricyclic compound or a pharmaceutically acceptable salt thereof according to claim 2, wherein Y^{a} is —OCH₂—.

5. The therapeutic agent for bladder hypersensitivity comprising, as an active ingredient, a tricyclic compound or a pharmaceutically acceptable salt thereof according to any of claims 2 to 4, wherein R¹ is a hydrogen atom, substituted or unsubstituted lower alkoxy or halogen.

6. The therapeutic agent for bladder hypersensitivity comprising, as an active ingredient, a tricyclic compound or a pharmaceutically acceptable salt thereof according to any of claims 2 to 4, wherein R¹ is a hydrogen atom.

7. The therapeutic agent for bladder hypersensitivity comprising, as an active ingredient, a tricyclic compound or a pharmaceutically acceptable salt thereof according to any of claims 2, 5 and 6, wherein Y^{a} is —CH₂SO₂—, —SO₂CH₂— or —OCH₂—.

8. The therapeutic agent for bladder hypersensitivity comprising, as an active ingredient, a tricyclic compound or a pharmaceutically acceptable salt thereof according to any of claims 2, 5 and 6, wherein Y^{a} is —CH₂SO₂— or —SO₂CH₂—.

9. The therapeutic agent for bladder hypersensitivity comprising, as an active ingredient, a tricyclic compound or a pharmaceutically acceptable salt thereof according to any of claims 2, 5 and 6, wherein Y^{a} is —CH₂SO₂—.

10. The therapeutic agent for bladder hypersensitivity comprising, as an active ingredient, a tricyclic compound or a pharmaceutically acceptable salt thereof according to any of claims 2 to 9, wherein X¹—X²—X³ is S—CR⁷═CR⁸ (wherein R⁷ and R⁸ have the same significances as defined above, respectively).

11. The therapeutic agent for bladder hypersensitivity comprising, as an active ingredient, a tricyclic compound or a pharmaceutically acceptable salt thereof according to any of claims 2 to 9, wherein X¹—X²—X³ is CR⁵═CR⁶—CR⁷═CR⁸ (wherein R⁵, R⁶, R⁷ and R⁸ have the same significances as defined above, respectively).

12. The therapeutic agent for bladder hypersensitivity comprising, as an active ingredient, a tricyclic compound or a pharmaceutically acceptable salt thereof according to any of claims 2 to 11, wherein R^{2a} is formula (II): 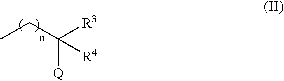
(wherein n, R³, R⁴ and Q have the same significances as defined above, respectively).

13. The therapeutic agent for bladder hypersensitivity comprising, as an active ingredient, a tricyclic compound or a pharmaceutically acceptable salt thereof according to claim 12, wherein n is 0.

14. The therapeutic agent for bladder hypersensitivity comprising, as an active ingredient, a tricyclic compound or a pharmaceutically acceptable salt thereof according to claim 13, wherein R³ is methyl, R⁴ is trifluoromethyl, and Q is hydroxy.

15. The therapeutic agent for bladder hypersensitivity comprising, as an active ingredient, a tricyclic compound or a pharmaceutically acceptable salt thereof according to claim 2, wherein R¹ is a hydrogen atom, Y^{a} is —CH₂SO₂—, X¹—X²—X³ is S—CR⁷═CR⁸ (wherein R⁷ and R⁸ have the same significances as defined above, respectively), and R² is formula (III):

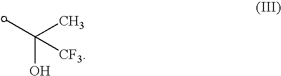

16. A therapeutic agent for bladder hypersensitivity comprising, as an active ingredient, a tricyclic compound represented by formula (Ib): 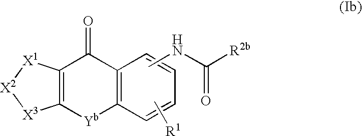
[wherein R¹ and X¹—X²—X³ have the same significances as defined above, respectively;
Y^{b} represents —CH₂O—, —CH₂S—, —CH₂SO—, —CH═CH— or —(CH₂)ₚ— (wherein p has the same significance as defined above); and R^{2b} represents formula (III) 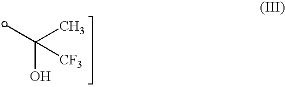
or a pharmaceutically acceptable salt thereof.

17. The therapeutic agent for bladder hypersensitivity comprising, as an active ingredient, a tricyclic compound or a pharmaceutically acceptable salt thereof according to claim 16, wherein X¹—X²—X³ is CR⁵═CR⁶—CR⁷═CR⁸ (wherein R⁵, R⁶, R⁷ and R⁸ have the same significances as defined above, respectively) or CR⁵═CR⁶CR⁷═N (wherein R⁵, R⁶ and R⁷ have the same significances as defined above, respectively).

18. The therapeutic agent for bladder hypersensitivity comprising, as an active ingredient, a tricyclic compound or a pharmaceutically acceptable salt thereof according to claim 16, wherein X¹—X²—X³ is CR⁵═CR⁶⁰ (wherein R⁵ and R⁶ have the same significances as defined above, respectively) or CR⁵═CR⁶—S (wherein R⁵ and R⁶ have the same significances as defined above, respectively).

19. The therapeutic agent for bladder hypersensitivity comprising, as an active ingredient, a tricyclic compound or a pharmaceutically acceptable salt thereof according to claim 16, wherein X¹—X²—X³ is 0CR⁷═CR⁸ (wherein R⁷ and R⁸ have the same significances as defined above, respectively) or S—CR⁷═CR⁸ (wherein R⁷ and R⁸ have the same significances as defined above, respectively).

20. The therapeutic agent for bladder hypersensitivity comprising, as an active ingredient, a tricyclic compound or a pharmaceutically acceptable salt thereof according to any of claims 16 to 19, wherein Y^{b} is —CH₂O—.

21. The therapeutic agent for bladder hypersensitivity comprising, as an active ingredient, a tricyclic compound or a pharmaceutically acceptable salt thereof according to any of claims 16 to 19, wherein Y^{b} is —(CH₂)ₚ— (wherein p has the same significance as defined above).

22. The therapeutic agent for bladder hypersensitivity comprising, as an active ingredient, a tricyclic compound or a pharmaceutically acceptable salt thereof according to claim 21, wherein p is 0.

23. The therapeutic agent for bladder hypersensitivity comprising, as an active ingredient, a tricyclic compound or a pharmaceutically acceptable salt thereof according to claim 21, wherein p is 2.

24. The therapeutic agent for bladder hypersensitivity comprising, as an active ingredient, a tricyclic compound or a pharmaceutically acceptable salt thereof according to any of claims 16 to 19, wherein Y^{b} is —CH═CH—.

25. The therapeutic agent for bladder hypersensitivity comprising, as an active ingredient, a tricyclic compound or a pharmaceutically acceptable salt thereof according to any of claims 16 to 19, wherein Y^{b} is —CH₂S— or —CH₂SO—.

26. Use of the tricyclic compound or the pharmaceutically acceptable salt thereof according to any of claims 1 to 25 for the production of a therapeutic agent for bladder hypersensitivity.

27. A method for treating bladder hypersensitivity, comprising a step of administering an effective amount of the tricyclic compound or the pharmaceutically acceptable salt thereof according to any of claims 1 to 25.
